(19) United States
(12) Patent Application Publication
(10) Pub. No.: US 2011/0302051 A1
(43) Pub. Date: Dec. 8, 2011

(54) MOBILE SYSTEM AND METHOD FOR INVENTORYING AND PURCHASING GOODS

(75) Inventor: Mehmet Arbatli, Houston, TX (US)

(73) Assignee: MediSouth, LLC, Houston, TX (US)

(21) Appl. No.: 13/114,630

(22) Filed: May 24, 2011

Related U.S. Application Data
(60) Provisional application No. 61/348,478, filed on May 26, 2010.

(51) Int. Cl. G06Q 30/00

(52) U.S. Cl. 705/26.8

(57) ABSTRACT

A system for gathering, storing, and updating inventory and product information in real time from a shared, centrally located database through a software application, such as a browser based application. This method generally includes the step of collecting product and inventory data through the use of a mobile device. The transfer of information can be through a Wi-Fi hot-spot, 3G, WiMax or 4G connection or other wireless network. The information will be gathered and automatically transmitted from a hand held device such as a barcode scanner, RFID reader, and the like, to a mobile device using a wireless connection technology, such as Bluetooth™ technology. The gathered information is then transferred to and from the database in real-time as a user manipulates the information within the database. Data will only be stored in the shared, centrally located database. Access to the data by the user will be allowed through an interface, such as a web based interface. The gather information can be used by a third party to order new inventory or resupply existing inventory. Collective purchasing can be accomplished based on the needs of the various users that access the system.

### CROSS-REFERENCE TO RELATED APPLICATIONS

This application claims the benefit of U.S. Provisional Application No. 61/348,478 filed on May 26, 2010, which application is hereby incorporated by reference for all purposes in its entirety.

### STATEMENT REGARDING FEDERALLY SPONSORED RESEARCH OR DEVELOPMENT

N/A

### REFERENCE TO MICROFICHE APPENDIX

N/A

### BACKGROUND OF THE INVENTION

1. Field of the Invention

The system relates to the collection and manipulation of data within a shared database through mobile and hand held devices. Specifically, the manipulation and analysis of business, inventory and product information stored within the shared database. Based on the information, a third party can analyze the shared information in the database to purchase additional inventory at a reduced cost to the various users based on collective purchasing power.

2. Description of the Related Art

Within various industries, including the medical community, inventory management and control have been an ongoing issue. Current technologies allow the user to scan the UPC of a product, and store the information within the memory of the scanning device. The device must then be connected to a computer, via a wired or wireless connection, in order to transmit the information to the software that is installed on that specific computer, eventually flowing into a database which is the storage backend to the software solution.

One problem with the current technology is that the user cannot get accurate feedback while scanning products and therefore must trust the hand-held devices to store accurate inventory and product information. The devices used either do not display any product information, or only display a limited amount of information pertaining to the scanned product. The said lack of feedback and usability results in inaccuracies which can render this inventory tracking method effectively unusable.

In addition to this, in prior systems, the user must also connect these hand-held devices to a software system located on a computer; which is another obstacle that must be overcome by the user to see the latest state of their inventory. This lack of feedback can also cause inaccuracies.

Another problem faced by the user with prior systems, which is often considered a major factor, is the cost of implementing inventory software into their business. Most small and medium sized businesses can not afford to invest in such software and hardware solutions. This cost barrier leads these businesses to manage their inventories manually, and causes time, money and productivity.

Therefore a low cost and easy to use solution is needed in the medical industry that will allow a business to access, maintain, manipulate, analyze, and order inventory without the prerequisite of building and managing proprietary and costly software and hardware solutions. In addition, a need exists where the inventory can be purchased at a reduced cost as a result of the inventory information.

### BRIEF SUMMARY OF THE INVENTION

The primary objects of the system are as follows:

1. Allow users or members, such as, but not limited to, medical service providers, physician groups and hospitals, access to a shared database via a network, such as the Internet, where information can be manipulated without a locally installed software based application on the user's computer.

2. Allow members automated ordering of replenishment stock from suppliers through the database upon the implementation of minimum inventory numbers by the user.

3. Allow specific network channels for medical supply companies' access to their customers for the purpose of replenishment of stocks.

4. Allow members access to the database through a software application, such as a web based application.

5. Allow a third party to monitor market data and trends of products stored in database by different criteria such as, but not limited to region, type of products and type of customers. Further analysis of said data by the third party to monitor and provide statistical analysis and trend data to related parties with the conditions which will be set forth by the third party in the future.

6. Monitor specific product purchase price information from members, allowing the third party to locate high prices and low prices paid for the products.

7. A third party having access to the information by various users or members to collectively purchase items for the users or members based on the inventory needs of the users or members.

8. Notification of members who are paying high prices for items that are managed in the database, alerting them where they may purchase items for a lower price.

### BRIEF DESCRIPTION OF THE DRAWINGS

In order that the manner in which the above recited and other advantages and objects of embodiments of the invention are obtained, a more particular description of the invention briefly described above will be rendered by reference to specific embodiments thereof that are illustrated in the appended drawings. Understanding that these drawings depict only typical embodiments of the invention and are not therefore to be considered to be limiting of its scope, the invention will be described and explained with the additional specificity and detail through the use of the accompanying drawings in which:

FIG. 1 illustrates a high-level view of the claimed invention and its most elementary physical components.

FIG. 2 is a schematic representation of the system which depicts the user interfaces and the main logical modules which comprise the claimed invention.

FIG. 3 is a flow diagram of the claimed invention describing the multiple steps that the customer needs to take to find and add products to the customer product list.

FIG. 4 is a flow diagram of the system in which the invention is practiced for one of its primary functions, the management of customer product inventory.

FIG. 5 is a block diagram of the system of the claimed invention.

FIG. 6 is a screen shot of an Update Inventory Page.

FIG. 7 is screen shot of Product Information Page.

FIG. 8 is a screen shot of Inventory Report Page.

FIG. 9 is a screen shot of Add Product Page.

Before discussing the figures, a brief description of the claimed invention is disclosed.

### DESCRIPTION OF SYSTEM

The system relates to the collection and manipulation of data within the shared database through mobile and hand-held devices. Specifically, the manipulation and analysis of business, inventory and product information stored within the shared database.

The claimed invention allows a user to log in to his/her/its account which resides in the shared database. In the preferred embodiment, an application which runs on an Internet-enabled mobile device and updates data in real time. The product and inventory data does not need to be stored on the mobile device locally. The application does not necessarily need to be a native application which runs on the mobile device. Instead, the manipulation and management of inventory and product data can be made through a web application, which can be used with a web browser application on the mobile device.

Database Site:

The centrally located database and database management system may have various architectures, such as but not limited to, relational, network, flat and hierarchical databases and database management systems. For illustrative purposes only, one embodiment disclosed herein will be described with respect to a relational database and a relational database management system.

The database platform will allow all users or members, such as various separate businesses access via a network (e.g., the Internet). The user can hold inventory information and monitor inventory in real time using a display application, such as a browser. This database will typically hold all encompassing information of products inputted. Examples of information of products, include but not limited to, UPC codes, product descriptions, pictures of the products, quantity, sizes, manufacturers, distributors and other related information. The database will also hold all member business information.

Customer Site:

Various businesses (sometimes referred to as a party, company or user) interested in using the system will generally apply for use through a third party, such as MediSouth, LLC, of Houston, Tex. and once approved the company will be sent a specifically paired input device (e.g., a Bluetooth™ scanner) and a mobile device (such as an iPad™ device or Android™ device), as well as a user name and password to obtain access to their individualized account. Once the user receives their devices all the users will need is a network at their location for functionality. Such networks include a WAN or LAN, (e.g., a Wi-Fi hot spot or 802.11 network or other wireless communications network). Besides the mobile device and the software on the mobile device for accessing, transmitting, receiving, inputting and displaying information, there is no other device or software needed by the user to gain or maintain access to the database, therefore eliminating software and software maintenance costs.

Once a user receives the equipment (e.g., the input device and the mobile device), user name, and password they are ready to enter their user account. Once the user has entered their account they will have multiple pages within their account such as a page for adding products, view inventory, settings, and orders.

Within the add products page, the member will input their existing inventory information into their account from our database information. From there the user will scan any incoming product and have an option to add it to their inventory, as well as scan any outgoing product to remove it from their inventory. The information scanned (product information code or identifier) is then transported over the Internet to our servers. This information is used to manipulate and update the inventory data of the member within the database. Once the manipulation is complete, the response is sent back over the Internet to the mobile device, allowing the member to monitor activity levels in real time. If for some reason the product is not found, the user may update the database with a manual input of data. Business specific information may be entered into the system as well, including supplier and purchase price which can allow members to receive comparison and competitive pricing information in the marketplace.

Within the inventory page the user will have the ability to view, print and send partial inventory information or entire inventory information. The user may implement customized minimum inventory numbers to their re-order account settings, and the third party will automatically trigger shipment of that product, when such numbers are reached. The user may set a quantity of the product desired once the minimum inventory number is triggered. This system therefore eliminates the need to manually maintain and order the replenishment of inventory. If the user wishes, they may opt out of automatic ordering and use the system only to manage their inventory, and manually order by phone or online.

The users of the system will not be restricted to managing solely the inventory purchased from the third party, and will have the ability to add product information and to manage the inventory of products purchased from other medical supply companies. The automatic ordering system can be open to the third party's competitors as well, through specific network channels based on a subscription model that will be set by the third party. The competitor company may pay a fee in order to access the system and utilize their specific customer purchasing information, allowing the user automatic ordering from specific medical supply companies as well.

Within the orders page, the user will have access to their order history and any orders that may be pending. The users may also view a real time tracking of their purchased products through a tracking icon. This icon will allow them to view the status of their purchase from warehouse processing, to delivery status.

The system will also have the ability for offices to monitor their supplies, such as pharmaceutical samples, office supplies, and implement customized minimum inventory numbers. The user can input the email address of their sales representatives, and when the customized number is reached an email can be automatically sent to the representative notifying them more samples are needed. This will not only maximize (or make efficient) the day of the representative, but also maximize exposure of manufactures to the users' offices.

Data Processing Site:

A shared database will store a variety of pertinent information, and a third party, such as MediSouth, LLC, will process this information through statistical data collection. As a result, the third party will have the ability to:

1. Analyze market data including, but not limited to, geographical distribution of various product categories, pricing and manufacturers.

2. Provide market research data to distributors, manufacturers, government and non-profit organizations.

3. Perform trend analysis and compile and organize periodical reports that may be distributed on a subscription basis based on rates that will be set by the third party

With this system, the third party will have access to all account information stored within its database. Therefore, the third party will have the ability to monitor all users' purchase prices by city and state. This may result in a number of advantages for the user, such as time and cost reduction in buying and planning areas of their business. If a user is purchasing a specific product over the average market value, they will have the opportunity of receiving notification of a lower price that is located by the third party. This typically allows every office of the user which utilizes the system to be assured that they are receiving the best price known for a specific product. The third party generally will hold all rights and privileges to any and all information of its users.

This system will also enable the third party to monitor the amount of specific product being used by each user from user's inventory. A product list for each business can be complied creating a purchasing pool. The product list (or needed items list) can be generated by a trigger event (such as when the inventory for a product is below a threshold of items needed by the user). This will empower the user to monitor usage trends of products by its personnel, granting the third party pertinent distribution data, allowing the third party greater negotiation and purchasing power (sometimes known as collective purchasing). The third party is then able to pass these savings down to the user.

The claimed invention will change the way the supply market (e.g., the medical industry) is currently conducting business, by redirecting cost savings to the businesses that purchase and use the supplies, at the same time giving peace of mind, and essential inventory organization tools needed on a day to day basis.

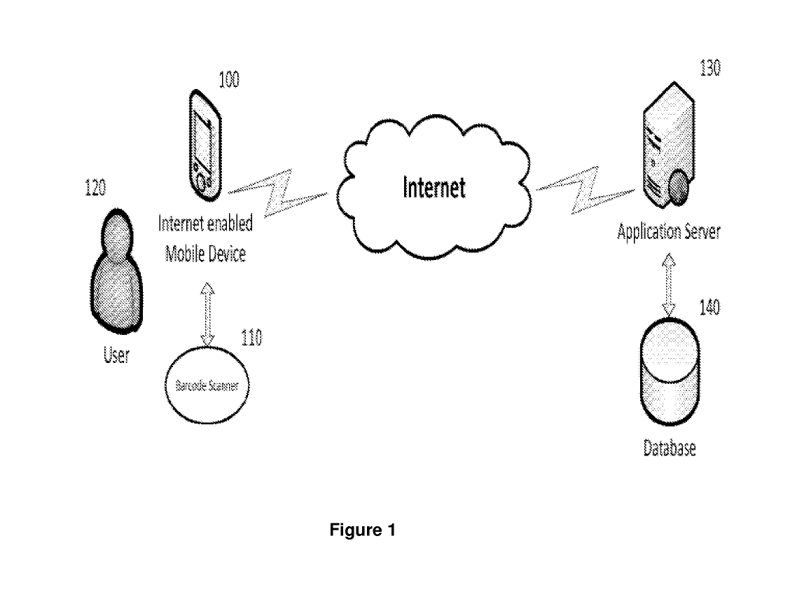

FIG. 1 and the following discussion are intended to provide a brief, general description of a suitable computing environment in which the invention may be implemented and used. The claimed invention allows various types of mobile devices to be used for inventory management, the only requirements being a network (such as the Internet) connection, and capability of pairing the mobile device with an input device, such as a barcode scanner. Therefore, the embodiments of the claimed invention may comprise many different types of mobile devices and input devices.

With reference to FIG. 1, an exemplary system for implementing the preferred embodiment includes a user (or client) side and a server (or host) side. On the user side, a general purpose mobile communication device 100, and an input device, such as a barcode scanner 110 which is paired to mobile device 100. In one embodiment, the computing device 100 is an iPad device, manufactured by Apple, Inc. and the input device, is a 7× and 7×Rx scanner, manufactured by Socket Mobile, Inc. The mobile device 100 should preferably have a connection to the Internet over a Wireless Internet connection. EDGE, 3G or any other type of mobile Internet connection standard can also be used, although depending on the type of connection, the standard might cause a reduction in the responsiveness of the mobile application since mobile Internet connections are generally prone to higher network latencies, and depending on the connection standard, lower data transfer speeds.

On the server end of the system, there exists an Application Server 130 which contains the modules and business logic which is used to process and respond to product and inventory management requests, and a Database Server 140 which is used to store all product, account and inventory data. The Application Server 130 is a logical labeling and can actually be comprised of multiple servers depending on the needs of the specific embodiment of the invention. Application Server 130 should have layers for security, authentication, load balancing and other considerations. For back up and redundancy purposes, the servers can be located in different geographic areas. The Database Server 140 in question includes the data store, and the Database Management System which handles all data requests to the server. The Database and Database Management System comprising Database 140 in FIG. 1 may have various architectures, such as but not limited to, relational, network, flat and hierarchical databases and database management systems. For illustrative purposes only, the preferred embodiment disclosed herein will be described with respect to a relational database and a relational database management system. The Application Server 130 can also have many architectures and can be distributed over many distinct devices based on the performance requirements of the specific implementation of the system.

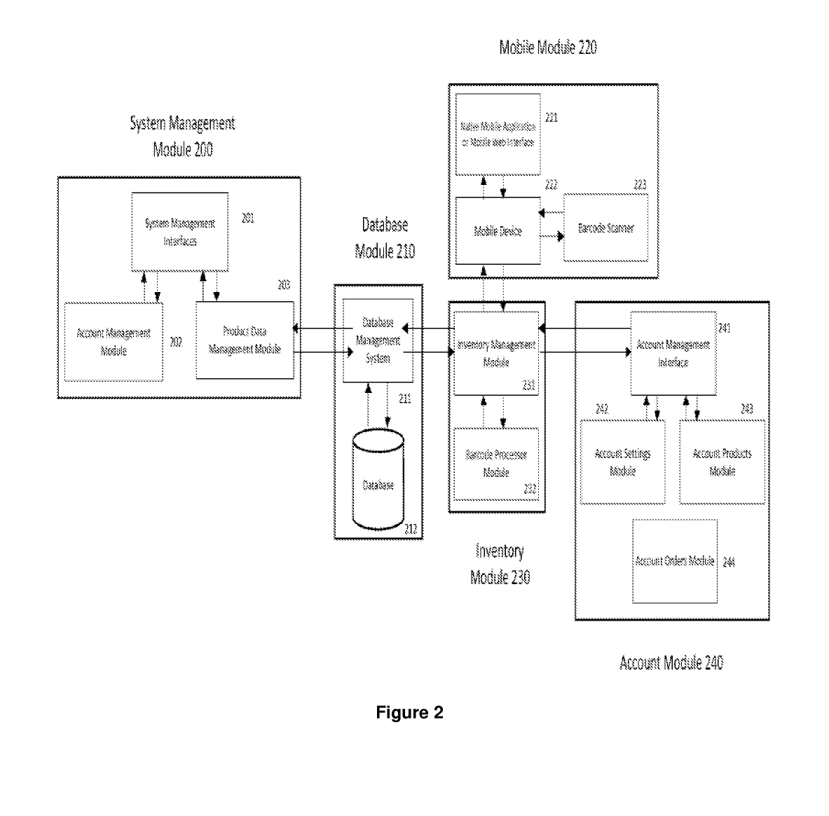

FIG. 2 illustrates the main interfaces and modules which comprise the preferred embodiment. This illustration is made from a relatively lower level standpoint than the one in FIG. 1. Among the logical modules is the System Management Module 220 which contains the System Management Interfaces 201, the Account Management Module 202, and the Product Data Management Module 203. The interfaces 201 are a set of user interfaces used by the administrators of the system to do many tasks among which are the management of customer accounts, the management of product and product packaging information, and the analysis of price and consumption data which will be described later. The interfaces 201 may be in many formats, such as but not limited to, native interfaces written for a specific computer architecture, or web based interfaces viewable through a browser which runs on a computer, mobile device or any other type of device. The main requirement is network (e.g., Internet) connectivity, so that a connection to the Application Server 130 and Database 140 can be maintained.

Module 202 is utilized by interfaces 201 to accomplish tasks related to the customers in the system which are to use it to manage their inventories. These tasks include, among others, the creation of a customer (sometimes referred to as a user) account, updating the data of the customer account, and generating various reports regarding the customer account. Module 203 is utilized by interfaces 201 to accomplish tasks related to the products in the system. One of the objectives of the invention is to store data in Database 140 of as many different products as possible. This data includes, but is not limited to, product type, product description, manufacturer, packaging information, product photograph and Universal Product Codes (UPC) for all types of packaging for the product. An advantage for storing this data is ease of use for the customers. During the initial setup phase of a customer account, which is discussed later, the customer needs to specify which products she wants to track inventory for. During this phase, if the customer cannot find a product in the database, she may have to manually enter the basic product and packaging information for that product. In order to reduce the possibility of this, a central database 212 should have all possible products that might be used by its customers.

Another high-level module depicted in FIG. 2 is the Database Module 210 which directly corresponds to module 140 in FIG. 2. This module is comprised of a Database 212 which maintains the data in the storage data structures, and a Database Management System 211 which includes multiple software applications that perform the function of obtaining and retrieving data from, and saving data to the database. Module 210 may have various architectures, such as but not limited to, relational, network, flat and hierarchical databases and database management systems.

Another high-level module depicted in FIG. 2 is the Mobile Module 220 which directly corresponds to module 100 in FIG. 2. This module is comprised of the Mobile Device 222, the Native Mobile Application or Mobile Web Interface 221 which the customer interacts with, and the Barcode Scanner 223 which is used to scan or input product identifiers, such as UPCs and transfer them to device 222. The present invention disclosed herein will be described with a Bluetooth connection but any other type of connection between device 222 and device 223 is acceptable as long as the scanned UPCs are transferred to the mobile device. Interface 221 could be a set of interfaces developed for a specific type of mobile device, or could be implemented as a web application usable through a web browser which runs on device 222.

Inventory Module 230 is another high-level module depicted in FIG. 2. This module is responsible for the management and manipulation of inventory data as customers use module 220 to manage their inventories. This module is made up of the Inventory Management Module 231 which consists of the business logic for the management of inventory, and the Barcode Processor Module 232. Many product package barcodes have UPCs which are universally standardized, such as for medical products, the EAN or the GTIN standards. Some manufacturers and suppliers choose to identify some products with more advanced barcodes which contain not only the Universal Product Code of the product but also extra information such as for medical products, the best before date, as there are many different types of medical products with different usage and storage needs. In order to make sure that there is only one record in Database 210 for each product in the system, when a barcode is scanned with scanner 223 and is sent to a module for processing, the barcode needs to be processed in case the barcode contains more than just the UPC of the product. In this case, the UPC should be extracted from the barcode and used as the product identifier, otherwise many different records for a single product might end up being saved and used within the system, which could be detrimental to the inventory management and information analysis capabilities of the system.

The Account Module 240 is the latest high-level module of the invention, and is comprised of the interfaces and modules which the customer will utilize to complete tasks such as specifying product lists, generating inventory reports, and managing orders. This module consists of the Account Management Interface 241, the Account Settings Module 242, the Account Products Module 243, and the Account Orders Module 244. The Account Management Interface 241 is a set of interfaces with which the customer will interact with. These interfaces may be implemented for a specific device architecture, or might be web based, and viewable through a web browser. Interface 241 will use modules 242, 243 and 244 to manipulate the database and complete customer tasks. Module 242 is responsible for the management of main customer account tasks. Module 243 is responsible for the management of customer product lists, product purchase prices and product consumption analysis. Module 244 is responsible for the management of customer orders. All manual orders the customer makes as well as the automatic orders based on cutoff numbers will be managed through module 244. This module will also be responsible for communicating with 3rd party suppliers which might be registered in the system with specific network channels.

Those skilled in the art will appreciate the fact that the claimed invention disclosed herein might be implemented with relatively different modular architectures, and the modules and embodiment described in this document are thought of as logical modules and do not directly correspond to any specific software. Also, those skilled in the art will also realize that this type of architecture can be implemented using any type of technology and method based on the specific needs of the system.

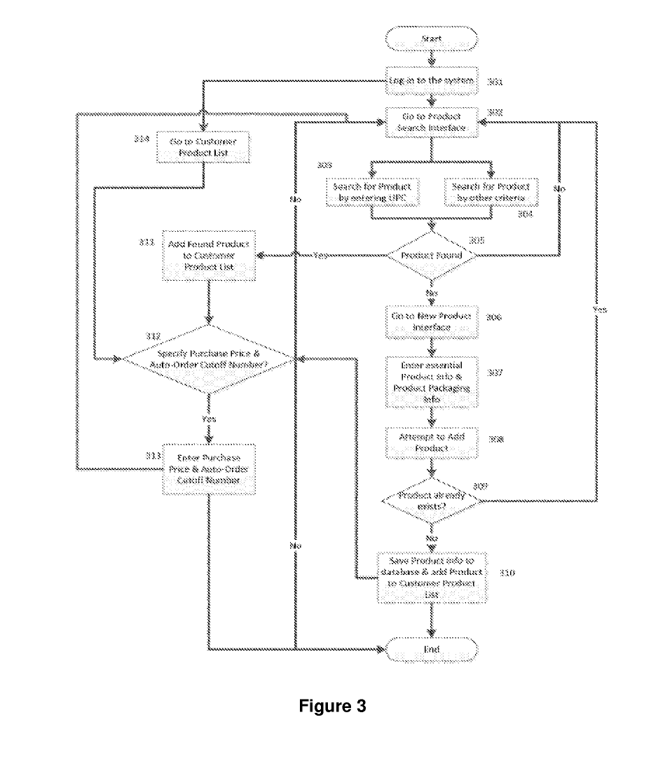

FIG. 3 is a flow diagram of the present invention describing the multiple steps that the customer needs to take in one embodiment of the present invention in order to find and add products to the customer product list. This diagram also depicts the optional steps that need to be taken by the customer to enter purchase price data, and to specify cutoff numbers and order quantities for automatic ordering of supplies. This flow diagram depicts the processes and methodology for manipulating data stored in module 210 through the use of modules 241, and 243 and to some extent, module 232.

A customer prepares module 243 in order to specify the products which she uses by first logging in to the interface 241, as depicted in block 301, which authenticates the customer with the specified credentials through database 210. As described previously, these credentials are given to the customer by the administrators of the system. The specification of customer credentials is made through interface 201 and module 202 and is stored in database 210. In the description of this embodiment of the system, all product and customer data are thought to be in one database module but that does not necessarily mean that all data must be stored in one physical database or one database instance. Those skilled in the art will recognize that this data can be shared over many database instances based on many criteria, such as but not limited to performance, fault tolerance and throughput. The module 210 described in this embodiment of the present invention is a logical module which may or may not contain these different functional separations.

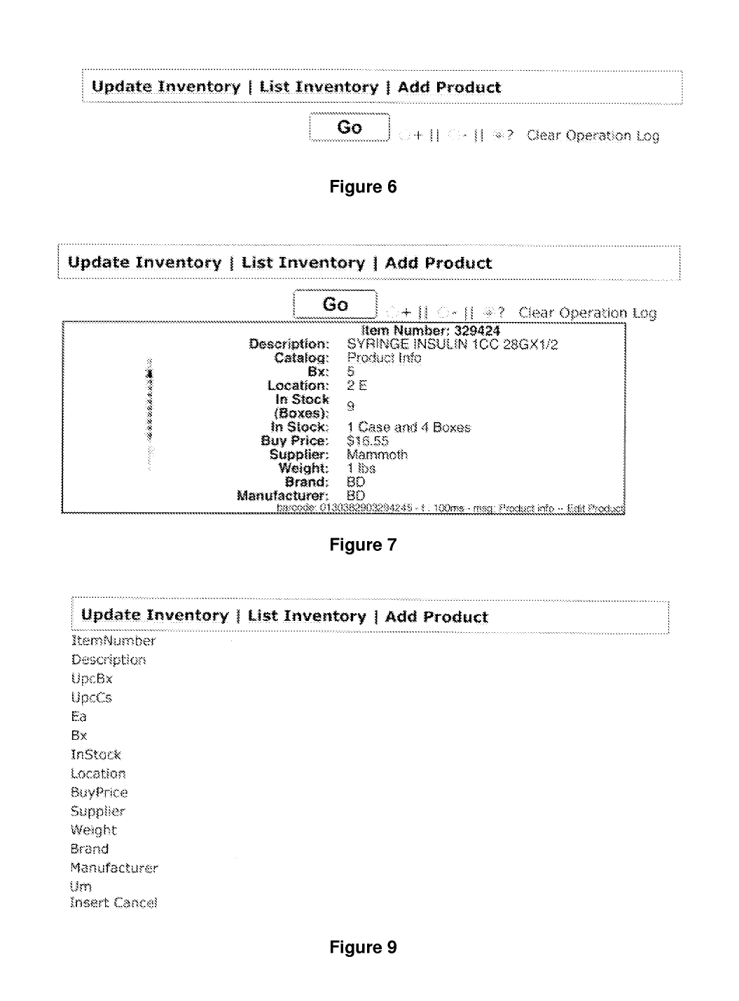

After the customer logs in, she will go to the Product Search section of interface 241, as depicted in block 302, from where she will attempt to search for the products that she wishes to track inventory for. FIG. 6 depicts a screen shot of the display on the mobile device of the system that allows access to the user. The search can be made based on many criteria such as but not limited to type of product, manufacturer, description, and specs, as depicted in block 304. One skilled in the art would recognize that the customer may choose to use interface 241 through the use of an Internet-enabled mobile device or through a desktop or notebook computer, among other options, she can pair scanner 223 to the present computer or mobile device and make the product search by scanning the barcode of the product she wishes to find, as depicted in block 303. In this case, the UPC which is sent to the system must first be processed by module 232 in order to extract the unique identifier of the product. When the search is made, the search results are transferred to interface 241.

The customer can choose to narrow her search criteria or to specify different criteria if too many results were returned by her search, or if she were not able to find her product in the search results. Among the possible outcomes of a product search, as depicted in decision block 305, is the customer finding the desired product, the customer not finding the desired product and choosing to refine the search criteria, going back to blocks 303 and 304, and the customer choosing to add the product to the database manually. FIG. 7 depicts a screen shot of product information for example, a syringe.

If the customer finds the desired product, she selects and adds the product to her product list as depicted in block 311. This selection and addition can be made in a number of ways depending on the specifics of interface 241. After adding the product to the product list, the customer can then choose to specify purchase price and automatic ordering cutoff quantities for the recently added product. This choice is depicted in decision block 312. If the customer chooses not to specify purchase price or cutoff quantities, she can either end the process, or go back to block 302 and search for another product to add to the list. If the customer does choose to add purchase price or cutoff quantity data for the product, she goes to the product detail screen of interface 241 and enters the desired data through the screen as depicted in block 313. This data is then stored in database 210. After adding purchase price or cutoff quantity data, the customer can choose to go back to block 302 and search for another product, or can end the product list management process.

If the customer cannot find the desired product, she will also have the choice of manually adding the product to database 210. This process, which is depicted in blocks 306, 307, 308, 309 and 310 adds the product data to database 210 using modules 243 and 203. FIG. 9 depicts a screen shot for a user to manually enter into the database 210 information about a product.

As depicted by block 307, the customer will be required to enter basic data about the product, such as but not limited to, manufacturer, manufacturer code, product type and product description. In addition to this basic data, the customer will also need to specify the basic packaging data for the product; for instance, as in how many items of the product is stored in one box, and how many boxes of the product is stored in one case. This packaging data may be required because manufacturers assign different UPCs for different types of packaging and the current invention requires that all UPCs of all types of packaging for a product exist in database 210, so that module 231 can uniquely identify scanned products in order to be able to manage and manipulate inventory data. Once product packaging data is also specified, the customer attempts to add the product to the database as depicted in block 308.

Before the product is added to the database, one last check is made to make sure that the customer is not inserting a duplicate product to database 210. This is not an unlikely possibility, since a customer might fail to find a product which exists in the database, especially if she is not doing the search with the product UPC using scanner 223. Manually entered criteria such as product type and product description might result in the customer not finding the product and thinking that it does not exist in database 210. The system takes this possibility into account and checks database 210 using product data such as manufacturer code or packaging UPCs which can uniquely identify a product in the database. If the product actually exists in database 210 and the customer has failed to find it in her searches, this checking algorithm will not add the duplicate product record to the database and instead will inform the customer of the situation. This is depicted in decision block 309. The customer can choose to add the existing product to her list and go to decision block 312 as discussed previously. If the final product check also does not come up with the product in database 210, this means that the product actually does not exist in the database and must be added, and adds the product with the data entered by the user. After this addition, which is made via module 203, the newly added product will also be added to the product list of the customer using module 243, and the customer will be given the option of specifying purchase price or cutoff numbers as discussed previously and depicted in decision block 312.

This product list manipulation process is thought to mostly occur when an account is initially defined in database 210, using interface 201 and module 202. When a customer first starts using the invention disclosed herein, she will be required to specify the list of products that are used frequently, since that is the main requirement for the management of inventory with the use of modules 220, and 230. Once the initial specification of the product list is complete, the customer will only return to the process described in FIG. 3 when the list of frequently used products changes, or when she wishes to specify or change automatic ordering cutoff quantities, or when the purchase price of one of the products in the product list changes. In these cases, the customer will go through some of the steps depicted in FIG. 3, based on the specifics of the situation. If the customer wishes to update the automatic ordering cutoff quantities or the purchase price of a product in the product list, she will again start in block 301, but instead of going to the product search interface depicted in block 302, she will go to block 314, where she will see a list of all products which she had already specified, select one of the products on that list, and then go to nodes 312 and 313 to enter or update this data. If the customer wishes to add another product to the product list, then she will start with block 301 and will continue through the steps of finding and adding a product to the product list that were discussed previously.

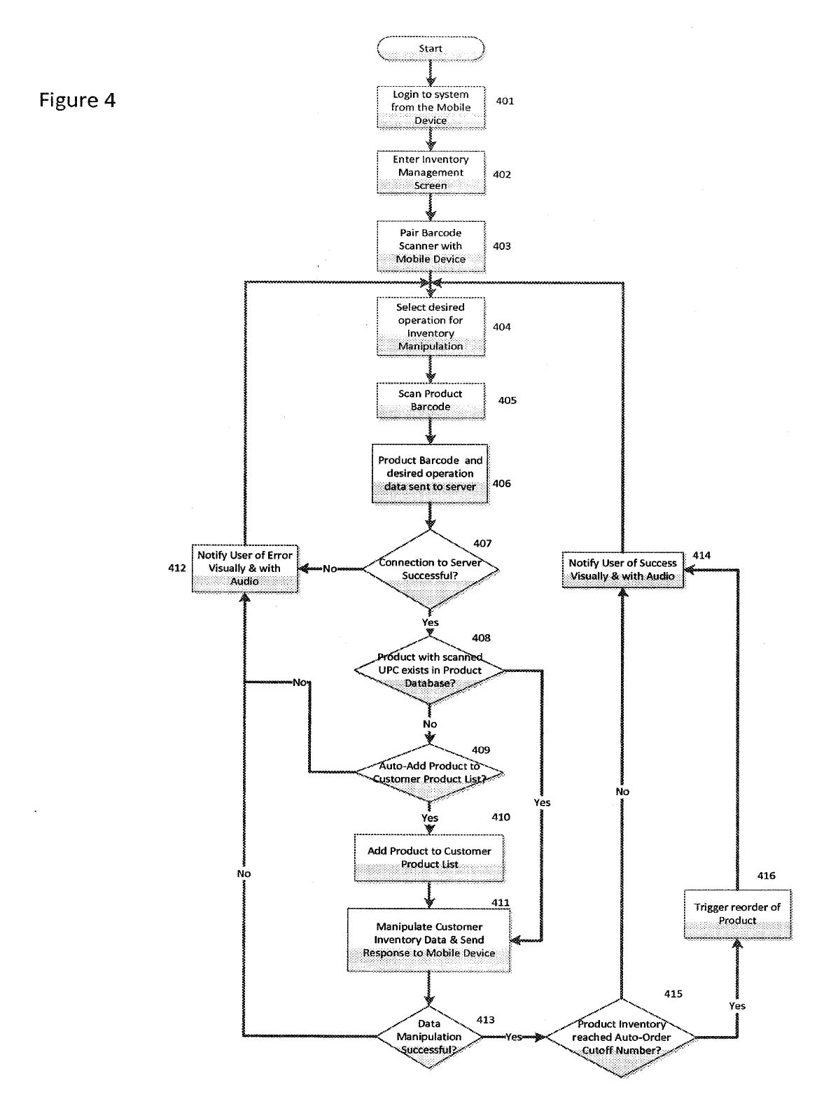

FIG. 4 is a flow diagram of the present invention describing the multiple steps that the customer needs to take in one embodiment of the present invention in order to manage inventory data. This operation is done using modules 220, 230, and 210. In the invention disclosed herein, inventory manipulation is done using the mobile device 222 which runs a native or web based application 221 designed for inventory management, and a barcode scanner 223 used to scan product UPCs. This configuration is not the only possible configuration for this operation but is the preferred configuration to give as much accuracy as possible during the inventory manipulation.

Similar to the process in FIG. 3, the customer needs to log in with the account credentials from the mobile device as depicted in block 401. Once the customer is authenticated by the system via module 202, the customer can then go to the inventory management screen of application 221, as depicted in block 402, from which the application will manipulate the database 210 using modules 231 and 232. Also, one must keep in mind that, as described previously, the customer can choose to log in to the system using an alternative inventory management application which runs on a desktop computer, as long as that application can connect to the Internet and communicate with database 210 via modules 231 and 232, and can receive scanned barcode information from the scanner 223. Another alternative is the customer using a web based inventory management interface to use the inventory management system. This web based interface could be the same interface that the customer can use via the mobile device, or can be a different kind of interface with different visual elements; the only requirement being that the application can communicate with database 210 via modules 231 and 232, and can receive scanned bar-code information from the scanner 223. Once the customer logs in to the system, she must make sure that the device that is being used is paired to scanner 223, as depicted in block 403, and is capable of receiving scanned barcode information.

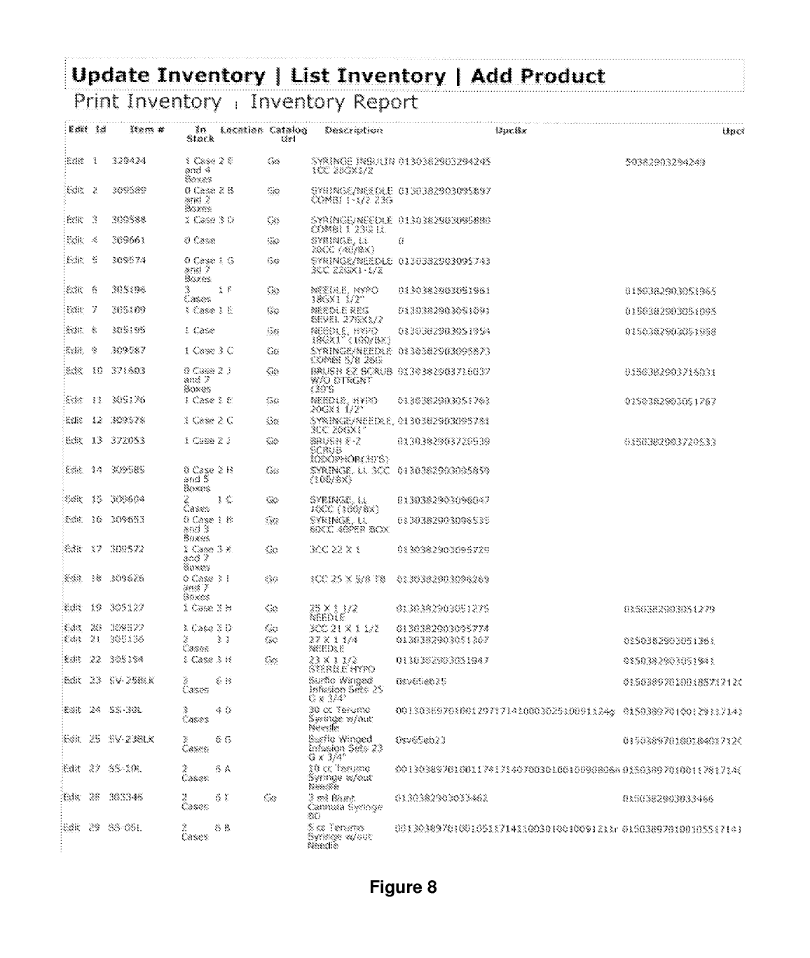

The invention disclosed herein is capable of many different kinds of operations with respect to inventory management, such as but not limited to, adding a unit of a product to the inventory, removing a unit of a product from the inventory, or reporting the current inventory state of a product in the inventory. For example, FIG. 8 depicts a screen shot of an exemplary inventory list.

In interface 221, the customer must first choose which of these or any other supported operations she wants to perform, as depicted in block 404. After selecting the type of operation, she then scans the barcode of a product with the scanner 223, as depicted in block 405. The scanned barcode information is transferred to the mobile application via the communication technology which was used to pair device 222 and the scanner 223. Once the application 221 receives the barcode from the scanner 223, the application 221 prepares and transmits the desired operation and the scanned barcode via Internet to the application server 130 which was depicted in FIG. 1. This transmit operation is block 406 in FIG. 4. During this transmit phase, there is a possibility that a connection with server 130 is not established, which might be due to a number of device and network related reasons. This situation is depicted in decision block 407 in FIG. 4. If a connection with the server 130 is not established after a previously specified amount of time has passed, the customer is notified visually and with audio as depicted in 412. If the connection was successful, and the barcode and operation data was transmitted to the server 130, then modules 231 receives the data. Module 231 sends the barcode data to module 232, so that the UPC of the product can be extracted from the scanned barcode. One needs to keep in mind that only some of the medical products targeted by this invention carry more information than the product UPC on the barcodes. Based on barcode standards such as GTIN or EAN among others, module 232 processes the barcode and extracts the UPC of the product, returning it to module 231. If module 232 fails to extract the UPC from the barcode for any reason, such as a malformed barcode, it notifies module 231 and an error response is sent back to application 221. If the UPC is extracted successfully, module 231 manipulates database 210 based on the inventory operation selected by the customer. If the customer has selected to increase the inventory of the scanned product, then module 231 manipulates database 210 accordingly. Similarly, if the customer has selected to decrease the inventory of the scanned product, then module 231 manipulates accordingly. If the customer has only wished to see the current inventory state of the scanned product, then no manipulation of database 210 is made. Instead, module 231 only retrieves the inventory data of the scanned product. Generally, regardless of the inventory operation selected by the customer, module 231 retrieves the latest inventory state and basic product information of the scanned product from database 210 and sends it back to application 221 along with the results of the operation. The reason for this is that after every inventory operation, along with visual and audio feedback, this basic data and inventory state is also displayed to the customer to increase the quality of feedback of the invention to the customer. This way, the customer is certain at any moment of which product is being manipulated in the inventory, and can react immediately if an operation leads module 231 to make an erroneous manipulation in database 210.

During the manipulation operation, module 231 first checks to see whether a product with the specified UPC exists in database 210, as depicted in decision block 408. If no such product is found, an error response is returned to application 221, as depicted in block 412. If the product is found, then module 231 checks to see whether the specified product exists in the product list of the customer. If it does not exists in the product list, based on the account settings specified by the customer through interface 241 and module 242, module 231 can automatically add the product to the product list of the customer, or can take no action and return an error response to application 221, as depicted in decision block 409. If the scanned product is not in the product list of the customer, and the inventory operation is to decrease the inventory of the product, although the product can be automatically added to the product list of the customer in database 210, the decrease inventory action cannot be performed, since a newly added product must logically have an inventory of zero. In this case, the product is added to the product list, and then an error response is returned to application 221, as depicted in block 412, describing the nature of the problem. If the product exists in the product list of the customer with an inventory of zero, and the inventory operation selected by the customer is to decrease the inventory of the product, an error is returned to application 221 since module 231 cannot logically decrease the inventory of a product when the inventory is already zero. If the product is found, and the current inventory state is logically acceptable with respect to the specified inventory operation, then module 231 performs the operation, as depicted in block 411, updates database 210 and returns a success response to application 221 specifying the basic information about the product, the inventory state of the product before the manipulation of database 210 by module 231, and the inventory state of the product after the manipulation, as depicted in block 414. After manipulating inventory information in database 210, module 231 checks to see if the customer has previously specified an automatic reordering cutoff number, and if so, whether the resulting inventory quantity of the manipulated product has reached this cutoff number. This is depicted in decision block 415. As a result of this decision block, module 231 can choose to go directly to block 414 if a cutoff number was not specified, or product inventory has not reached the specified number. Alternatively, if a cutoff number has been specified and has been reached, module 231 will go to block 416 and trigger a reorder of the product. During this operation, module 243 will be used to retrieve the quantity of product that should be ordered, which was also determined by the customer while she was specifying her automatic reordering settings for the product, as described in the discussion of FIG. 3 previously. This automatic reordering is made by module 244. This reordering can be made directly to the third party, such as MediSouth. Alternatively, if the customer is buying the product from some other medical supply company, and if there is a an agreement between MediSouth and said supply company, the system can directly inform that supply company about the order using previously determined communication channels.

If module 231 cannot update database 210 as requested for any reason, as depicted in decision block 413, it returns an error response to the user, as depicted in block 412, with an error message specifying the nature of the error. This error can be caused by connectivity problems between application server 130, and database 140 (as depicted in FIG. 1), among many other possible causes depending on the nature of the specific embodiment of the invention. Since the invention disclosed herein is designed to work in real-time with an always-on Internet connection, each request is handled individually. As in all network based applications, the health of the connection between the client and the server cannot be guaranteed and a timeout period must be defined for all requests from the client to the server. This timeout period can be exceeded because of many reasons, such as but not limited to, the loss of the connection between the client and the server after the initial request has been sent, and the modules involved in the manipulation of inventory data taking too long to complete. With this in mind, an embodiment of the claimed invention can also choose to include offline capabilities to module 220 in which case a snapshot of the inventory state is stored locally on device 222. Under ideal conditions where each request receives a timely response, this snapshot would be updated along with database 210 to keep both data stores in sync. Under less than ideal conditions where there are connectivity problems between modules 100 and 130 (as depicted in FIG. 1), the inventory data that is stored locally on device 222 would be updated during the periods with lack of connectivity, and then would be synchronized with database 210 once the connection was restored. This way, the customer would not lose time and productivity in case, for instance, the Wi-Fi hotspot in her place of business went down for any reason. A mobile device which is capable of cellular data connectivity, such as but not limited to 3G or EDGE, would eliminate the need for such offline capabilities, since they work wherever there is cellular reception. While it is true that cellular data connections might result in relatively higher latencies than wireless Internet connections, they might be preferred to implementing offline capabilities in device 222, since synchronization would add a new layer of complexity to the embodiment of the invention, as well as creating the need to implement different versions based on the local storage capabilities of different mobile devices.

Those skilled in the arts will appreciate that depending on the architecture with which the claimed invention is embodied, many problems can occur between mobile devices and physical servers, and within software components, that cannot all be foreseen. It is of paramount importance that the user is notified of any error that has occurred with an easy to understand message which does not go too deeply into technical specifics, and that the personnel in charge of the maintenance of the invention is informed immediately so that the error can be resolved and the system remains usable buy all customers.

The description of the process pertains only to one embodiment of the claimed invention. Module 231 does not necessarily have to return such detailed information as depicted in blocks 412 and 414 after every inventory operation. It is only with the purpose of increasing feedback to the customer in order to increase inventory management productivity that the invention disclosed herein works in such a manner.

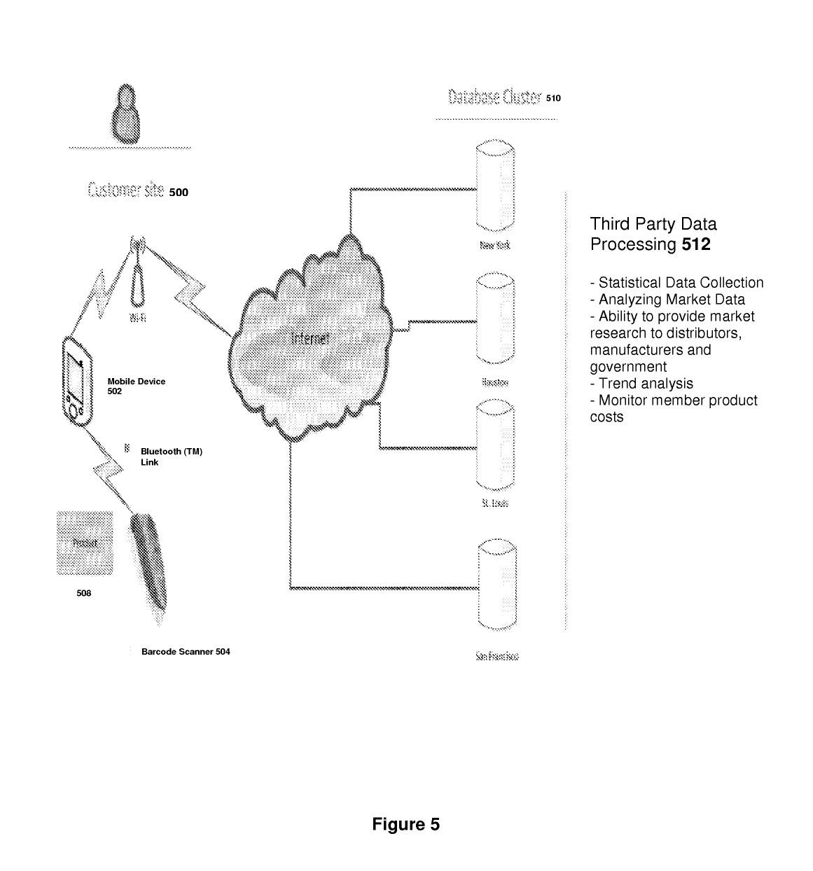

FIG. 5 illustrates a block diagram of the system of the claimed invention. As shown at the Customer Site 500, a mobile device 502 is connected to an input device (such as a barcode scanner) 504 via a wireless network, such as a Bluetooth™ wireless link 506. The input device 504 can input (or scan) information about a product 508 for uploading to the mobile device 502. Product information can then be transmitted to one or more databases in a Database Cluster 510. The databases of the Database Cluster 510 can be located in various locations, such as New York, N.Y., Houston, Tex., St. Louis, Mo. and San Francisco, Calif. A third party 512, such as MediSouth, LLC of Houston, Tex. can then process the information stored in the databases. Such processing can include, statistical data collection, analyzing market data, provide market research to distributors, manufacturers and government entities, trend analysis and monitoring of member product costs.

As previously mentioned, the goal for the claimed invention is to deliver a system for gathering, storing, and updating inventory and product information in real time from a shared, centrally located database through a browser based application. The claimed invention may be embodied in other specific forms without departing from its spirit or essential characteristics. The described embodiments are to be considered in all respects only as illustrative and not restrictive. The scope of the invention is, therefore, indicated by the claims rather than by the foregoing description. All changes which come within the meaning and range of equivalency of the claims are to be embraced within their scope.

I claim:

1. A system for gathering, storing, and updating inventory and product information in real time from a database for ordering products, comprising:
a computing device;
a plurality of input devices controlled by a plurality of parties, wherein the input devices are capable of reading identifiers of the products;
the computing device coupled to the database, the database consisting of product identifiers, inventory data and product information; and
the computing device coupled to the input devices via a network and information is retrieved from the input devices and stored in the database, wherein a collective purchasing list is generated for use in negotiating the purchase of the products for the plurality of parties.

2. The system of claim 1, wherein the network is the internet.

3. The system of claim 1, wherein the input devices are scanners.

4. The system of claim 3, wherein the scanners are barcode scanners.

5. The system of claim 1, wherein the parties are medical service providers.

6. The system of claim 1, wherein the computer device is an iPad operating system device.

7. The system of claim 1, wherein the computer device is an Android operating system device.

8. The system of claim 1, wherein the products consists of medical products.

9. The system of claim 8, wherein the medical products consists of syringes, pharmaceuticals or office supplies.

10. The system of claim 1, wherein the computing device is a web-based computing device.

10. A method of purchasing goods for third parties using a web-based system, consisting of the steps of:
inputting inventory information of product information from a plurality of third parties over a network;
storing said inventory information from the network in a shared database;
generating a order list of needed products based on the inventory information for the plurality of third parties,
creating a collective purchasing list from the inventory information of third parties; and
supplying said purchasing list to a supplier for the purchase of the needed products.

11. The method of claim 10, wherein the third parties consists of medical service providers, physician groups or hospitals.

12. The system of claim 10, wherein the inventory information consists of office supplies and medical products.

13. The system of claim 10, wherein the network is the Internet.

14. The system of claim 10, wherein the web-based system consists of an IPad operating system.

15. The system of claim 10, wherein the web-based system consists of an Android operating system.

16. A server used for purchasing products via a computer network comprising:
a database for storing inventory information for a plurality of users coupled to said server;
first means connected to said computer network for updating said inventory information via said computer network for the plurality of users, wherein said inventory information is compared against a trigger to create a needed item list;
second means for generating a needed list from the needed items; and
third means for ordering said needed items from a supplier.

17. The server of claim 16, wherein the plurality of users consists of medical service providers, physician groups and hospitals.

18. The server of claim 16, wherein the needed items consists of medical products and office supplies.

19. The server of claim 16, where the trigger is a minimum number of items provided by the plurality of users.

20. The system of claim 16, wherein a trend table is created from the inventory information.
